(19) United States
(12) Patent Application Publication
(10) Pub. No.: US 2025/0172480 A1
(43) Pub. Date: May 29, 2025

(54) INFORMATION PROCESSING METHOD AND INFORMATION PROCESSING SYSTEM

(71) Applicant: CANON KABUSHIKI KAISHA, Tokyo (JP)

(72) Inventors: SOTA TORII, Tokyo (JP); TETSUYA YANO, Ibaraki (JP)

(21) Appl. No.: 18/959,095

(22) Filed: Nov. 25, 2024

(30) Foreign Application Priority Data
Nov. 29, 2023 (JP) 2023-201983

(51) Int. Cl. G01N 15/075; G01N 33/543

(52) U.S. Cl. CPC G01N 15/075; G01N 33/54326

(57) ABSTRACT

The present disclosure provides a method for improving accuracy of calculating an amount of a target substance. A method for processing information includes acquiring an image including, a plurality of individual separated compartments, detecting presence of a target capturing substance in each of the individual separated compartments in the image, and calculating an amount of a target substance based on information regarding 1) the number of the individual separated compartments where the target capturing substance is detected and 2) the number of compartments in which the target substance is detected among the individual separated compartments in which the target capturing substance is detected and information regarding 3) the number of the individual separated compartments in which the target capturing substance is not detected and 4) the number of compartments in which the target substance is detected among the individual separated compartments in which the target capturing substance is not detected.

### BACKGROUND OF THE DISCLOSURE

### Field of the Disclosure

The present disclosure relates to an information processing method and an information processing system.

### Description of the Related Art

It is important to rapidly and accurately detect a target substance in a sample in fields such as medical diagnosis and academic research. For example, in the medical field, a target substance is detected and its concentration is measured based on an amount of a signal emitted from a reporter in an assay for a target capturing substance (containing an antibody and the like) that captures the target substance. In recent years, a high sensitive measurement method has been developed in which such a detection reagent is delivered to an individual separated compartment, the individual separated compartment is isolated using a hydrophobic solvent, and a reaction is detected using a fluorescence microscope or the like, as discussed in Japanese Unexamined Patent Application Publication No. 2013-521499.

<Issue that Information Processing Method According to Present Disclosure Solves>

In an antigen-antibody reaction, an antigen and an antibody are steadily in a bound state and a dissociative state, and a target capturing substance and a target substance are not necessarily bound to each other. Thus, it may happen that a dissociated target substance is filled into an individual separated compartment with no target capturing substance. However, according to Japanese Unexamined Patent Application Publication No. 2013-521499, only an individual separated compartment in which a target capturing substance is present is a measurement target, so that the number of target substances to be measured is underestimated.

### SUMMARY OF THE DISCLOSURE

The present disclosure is directed to the provision of a method for improving accuracy of calculating a concentration of a target substance in response to the above-described issue.

According to an aspect of the present disclosure, a method for processing information for detecting a target substance includes acquiring an image including, as an object, a plurality of individual separated compartments that contains: 1) at least one of the target substance and a target capturing substance that captures the target substance, or 2) neither the target substance nor the target capturing substance, detecting presence or absence of the target capturing substance in each of the individual separated compartments in the image, and calculating an amount or a concentration of the target substance based on information regarding 1) the number of the individual separated compartments in which the target capturing substance is detected and 2) the number of compartments in which the target substance is detected among the individual separated compartments in which the target capturing substance is detected and information regarding 3) the number of the individual separated compartments in which the target capturing substance is not detected and 4) the number of compartments in which the target substance is detected among the individual separated compartments in which the target capturing substance is not detected.

According to another aspect of the present disclosure, a calculation system includes a mounting unit configured to mount an aggregate that includes a plurality of individual separated compartments thereon, an image capturing unit configured to capture an image that includes the plurality of individual separated compartments as an object, and an information processing unit, wherein the information processing unit includes an image acquisition unit configured to acquire an image that includes, as an object, a plurality of individual separated compartments that contains: 1) at least one of a target substance and a target capturing substance that captures the target substance, or 2) neither the target substance nor the target capturing substance, a detection unit configured to detect presence or absence of the target capturing substance in each of the individual separated compartments in the image, and a calculation unit configured to calculate an amount or a concentration of the target substance based on information regarding 1) the number of the individual separated compartments in which the target capturing substance is detected and 2) the number of compartments in which the target substance is detected among the individual separated compartments in which the target capturing substance is detected and information regarding 3) the number of the individual separated compartments in which the target capturing substance is not detected and 4) the number of compartments in which the target substance is detected among the individual separated compartments in which the target capturing substance is not detected.

According to yet another aspect of the present disclosure, an information processing system including an information processing unit configured to process an image that includes a plurality of individual separated compartments as an object, wherein the information processing unit includes an image acquisition unit configured to acquire an image that includes, as an object, a plurality of individual separated compartments that contains: 1) at least one of a target substance and a target capturing substance that captures the target substance, or 2) neither the target substance nor the target capturing substance, a detection unit configured to detect presence or absence of the target capturing substance in each of the individual separated compartments in the image, and a calculation unit configured to calculate an amount or a concentration of the target substance based on information regarding 1) the number of the individual separated compartments in which the target capturing substance is detected and 2) the number of compartments in which the target substance is detected among the individual separated compartments in which the target capturing substance is detected and information regarding 3) the number of the individual separated compartments in which the target capturing substance is not detected and 4) the number of compartments in which the target substance is detected among the individual separated compartments in which the target capturing substance is not detected.

Further features of the present disclosure will become apparent from the following description of exemplary embodiments with reference to the attached drawings.

### BRIEF DESCRIPTION OF THE DRAWINGS

FIG. 1 is a flowchart illustrating an information processing method according to an exemplary embodiment of the present disclosure.

FIG. 2 is a cross-sectional view of a well plate according to the exemplary embodiment of the present disclosure.

FIG. 3 illustrates an example of an image with an aggregate as an object according to the exemplary embodiment of the present disclosure.

FIG. 4A illustrates an example of a bright field image according to the exemplary embodiment of the present disclosure.

FIG. 4B illustrates an example of a position image of an individual separated compartment according to the exemplary embodiment of the present disclosure.

FIG. 4C illustrates an example of a position image of a target capturing substance according to the exemplary embodiment of the present disclosure.

FIG. 4D illustrates an example of a position image of an individual separated compartment in which a target capturing substance is detected according to the exemplary embodiment of the present disclosure.

FIG. 5 illustrates an example of an information processing system according to the exemplary embodiment of the present disclosure.

FIG. 6 illustrates an example of a hardware configuration of the information processing system according to the exemplary embodiment of the present disclosure.

### DESCRIPTION OF THE EMBODIMENTS

Exemplary embodiments of the present disclosure will be described in detail below with reference to the attached drawings. However, components described in the exemplary embodiments are merely examples, and the technical scope of the present disclosure is defined by the scope of claims and not limited by the individual exemplary embodiments described below.

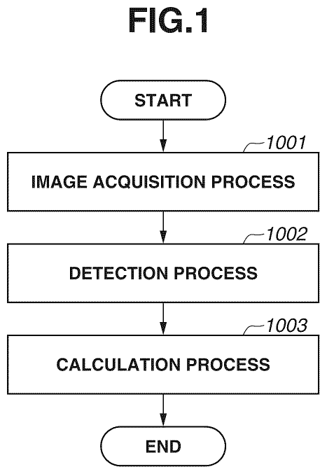

FIG. 1 illustrates an information processing method according to the present exemplary embodiment. The information processing method according to the present exemplary embodiment is an information processing method for detecting a target substance using an individual separated compartment and includes an image acquisition process, a detection process, and a calculation process. The individual separated compartment can contain at least either one of the target substance and the target capturing substance that captures the target substance.

In step S1001 (an image acquisition process 1001), an image is acquired that includes a plurality of individual separated compartments, which can contain the target substance, as objects. In step S1002 (a detection process 1002), a position of the individual separated compartment and the presence or absence of the target capturing substance are detected from the image acquired in step S1001. In step S1003 (a calculation process 1003), a concentration of the target substance is calculated based on information regarding the number of individual separated compartments in which the target capturing substances are detected and the number of individual separated compartments in which the target substances are detected among the individual separated compartments in which the target capturing substances are detected, and information regarding the number of individual separated compartments in which the target capturing substance is not detected and the number of individual separated compartments in which the target substances are detected among the individual separated compartments in which the target capturing substance is not detected.

As described in more detail below, the concentration of the target substance is calculated also using the information regarding the number of individual separated compartments in which the target capturing substance is not detected, so that a more accurate amount (number) or concentration can be calculated, particularly in a case where the target substance in low concentration is detected.

### <Individual Separated Compartment>

The individual separated compartment that can contain the target substance according to the present exemplary embodiment is described.

The individual separated compartment according to the present exemplary embodiment refers to an individual compartment that is independently separated from other individual compartments. A volume of the compartment is desirably very small, and is desirably 0.1 fL or more and 1000 fL or less. Desirable examples of the individual separated compartment may be a droplet and a well of a well plate. A target that includes a plurality of individual separated compartments may be referred to as an aggregate. For example, a well plate is an aggregate, and a liquid containing a plurality of droplets (which may include its container) is an aggregate.

As a droplet, a water-in-oil emulsion (W/O emulsion) is desirably used.

The above-described droplet can be prepared by a pumping method using an emulsion membrane.

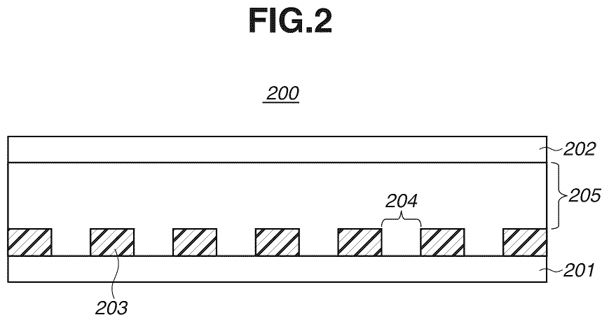

FIG. 2 illustrates an example of wells of a well plate according to the present exemplary embodiment. A well 204 is a concave portion that stores liquid and is separated from each other by a partition wall 203. The well 204 can have a bottom substrate 201 as a bottom surface, and a shape of an area surrounded by the bottom surface and side surfaces of the well 204 may be, for example, a cylindrical shape or a prismatic shape. In a case where the shape of the well 204 is a cylindrical shape with a circular bottom surface, it is desirable that a diameter of the bottom surface of the well 204 is for example, 0.5 μm or more and 2 μm or less, and a depth of the well 204 is 0.5 μm or more and 12 μm or less. It is further desirable that the diameter of the bottom surface of the well 204 is 1 μm or more and 9 μm or less, and the depth of the well 204 is 1 μm or more and 9 μm or less. It is desirable that an upper substrate 202 faces an opening of the well 204 and an upper surface of the partition wall 203 with a space 205 therebetween. The space 205 is a flow path through which various liquids flow, and each liquid can flow from an inlet to an outlet, which are not illustrated. A well plate may be a commercially available product such as a Simoa disc or may be fabricated.

The individual separated compartment is filled with a liquid containing the target substance, thus a sample is apparently concentrated, and it is possible to detect the target substance without an amplification process and to shorten a time for a signal to saturate. It is possible to set the volume of each of the individual separated compartments to be sufficiently small so that each compartment contains one molecule of the target substance or less, and a digital assay to calculate the concentration of the target substance in the liquid can be performed by counting the number of compartments from which a signal is acquired.

### <Target Substance>

The target substance according to the present exemplary embodiment is not particularly limited, and examples thereof include a protein, nucleic acid, a lipid, sugar, a low-molecule compound, an enzyme, a receptor, an antibody, an antigen, a cytokine, a hormone, and a membrane protein. The target substance according to the present exemplary embodiment may include at least one of a nucleic acid, an antibody, an antigen, an enzyme, and an enzyme substrate.

The individual separated compartment according to the present exemplary embodiment can contain a detection reagent, and a liquid containing the target substance can contain the detection reagent. The detection reagent desirably exhibits a signal by interacting with the target substance. The detection reagent can be any known one without limitation. For example, in a case where the target substance is a nucleic acid (hereinbelow, may be referred to as a target nucleic acid) and a clustered regularly interspaced short palindromic repeat (CRISPR)/Cas system is used, an effector protein, a CRISPR ribonucleic acid (crRNA) that binds to the target nucleic acid, and a reporter molecule can be used as the detection reagent. In other words, the crRNA binds to the target nucleic acid and activates the effector protein, and the activated effector protein modifies the reporter molecule to produce fluorescence as a signal. In a case where the target nucleic acid is used, various probes that generate signals by the presence of the target nucleic acid can be used without being limited to the above-described system. Here, generating a signal means generating fluorescence, a magnetic field, or the like, and the generated fluorescence, magnetic field, or the like can be detected as a signal using an appropriate detection unit. A probe can be used that changes a signal generated depending on an amount or concentration of the target nucleic acid.

Examples of the detection reagents in a case where the target substance according to the present exemplary embodiment is a protein include substances including an antibody, a modified antibody, an antigen, an enzyme, and an enzyme substrate aptamer, and these can generate signals by labeling with fluorescent dyes or using appropriate enzymes. Further, in a case where the target substance is an enzyme, an enzyme substrate can be used as the detection reagent. The enzyme substrate may be labeled with a fluorescent dye and modified by the substrate to produce fluorescent as a signal.

According to the present exemplary embodiment, the number of target substances per individual separated compartment is set to one molecule or less, so that an effective digital assay can be performed. Alternatively, in a case where the target capturing substance is used, the number of target substances per target capturing substance is set to one molecule or less, and thus a similar effect can be acquired and a digital assay can be performed.

### <Target Capturing Substance>

The target capturing substance according to the present exemplary embodiment is not particularly limited as long as it can capture the target substance according to the present exemplary embodiment. An example of the target capturing substance according to the present exemplary embodiment is a particle including a ligand. The particle may include a ligand in order to capture the target substance.

The ligand may include at least one of a nucleic acid, an antibody, an antigen, an enzyme, and an enzyme substrate. Examples of particles may include a polymer resin (styrene resin, acrylic resin, or the like) particle, a silica particle, an agarose carrier resin particle, a metal particle, a latex particle, and a magnetic particle. The target capturing substance is used, and thus the target substance can be separated using centrifugation or magnetism. Desirably, particles having a particle diameter of 1 μm or more and 10 μm or less can be used.

A method for filling (distributing) a liquid containing the target substance to the individual separated compartment according to the present exemplary embodiment is described.

A well plate and a well can be respectively used as an aggregate and an individual separated compartment according to the present exemplary embodiment as described with reference to FIG. 2. An aggregate 200 (here, a well plate) is left under reduced pressure to deaerate the space 205. Specifically, it is desirable to leave the well plate in a reduced-pressure desiccator at 0.1 atmosphere for a predetermined period of time. By deaerating, air is removed from inside the well 204, and a reaction liquid can be efficiently filled into the well 204. A deaeration time is not particularly limited and can be arbitrarily set. The method for filling the reaction liquid is not limited to a method using deaeration. Next, a hydrophobic solvent is delivered to the space 205 to seal it. In other words, the reaction liquid present in the space 205 above the well 204 is replaced with the hydrophobic solvent. As the hydrophobic solvent, for example, fluorine-based oil, saturated aliphatic hydrocarbon, unsaturated aliphatic hydrocarbon, aromatic hydrocarbon, and silicone oil can be used. Examples of fluorine-based oil may include Fluorinert, Asahiklin AE-3000 (manufactured by AGC Inc.), and Fomblin (manufactured by Solvay S.A.). Examples of saturated Hydrocarbons may include Isopar (manufactured by Exxon Mobil Corporation) and mineral oil.

According to the present exemplary embodiment, in a case where a droplet is used as the individual separated compartment, the individual separated compartment can be prepared by a pumping method using a shirasu porous glass (SPG) emulsion membrane with a liquid as a dispersed layer and an aliphatic hydrocarbon solvent containing a surface active agent as a continuous layer.

Signal generation according to the present exemplary embodiment is described. The individual separated compartment filled with the liquid containing the target substance appropriately performs signal generation in order to generate a signal.

For example, in an example using the above-described CRISPR/Cas system, incubation is performed at 37° C. The incubation promotes a CRISPR-Cas trans-cleavage reaction, resulting in generation of fluorescence derived from a fluorescent substance contained in a reporter molecule. In an example where the target substance is an enzyme, signal generation can be performed by promoting a reaction at an optimum temperature for the enzyme.

### <Image Acquisition Process>

Next, the image acquisition process is described. An image acquisition unit acquires an image including a plurality of individual separated compartments as objects. An image includes all information included in the image, for example, luminance, color, and shade for each coordinate, and the image acquisition unit can acquire necessary information from among all of these information pieces. The images may be extracted. The image may include the entire aggregate, namely the entire well plate or the entire liquid containing droplets in the container as the object or a part of the aggregate, namely a part of the well plate or a part of the liquid containing droplets as the object. It is desirable that the image includes at least either one of a bright field image and an image from which signal information indicating the presence of the target substance can be acquired. A fluorescence image can be an example of an image from which the signal information indicating the presence of the target substance can be acquired. The image acquisition unit is not limited as long as it can acquire an image, and may be a microscope integrated with an information processing system or an image capturing device such as a charge coupled device (CCD) camera, or a unit that is connected to these devices and acquires information of a captured image.

The image includes a particle and a well in the individual separated compartment. The image acquisition unit does not need to acquire an image in a single imaging and may perform imaging for a plurality of times and combine images into a single image.

In a case where the individual separated compartment is a well, a single image can include an entire image of a single aggregate, but a single image does not necessarily correspond to a single aggregate and may include a plurality of aggregates or only a part of the aggregate. For example, a commercially available microscope for imaging a well plate is known that images approximately 6×10³ wells in one shot, repeats imaging for 100 shots, and connects these shots to produce an image of 6×10⁵ wells.

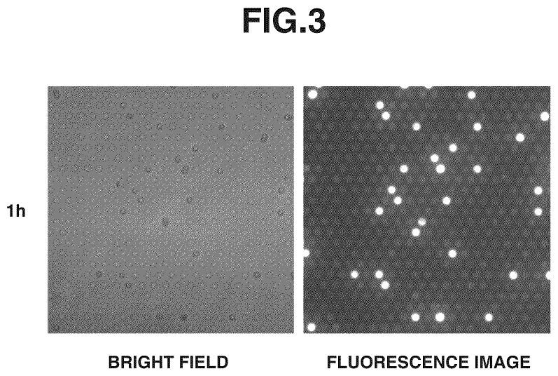

FIG. 3 illustrates examples of images including an aggregate as an object according to the present exemplary embodiment. FIG. 3 illustrates images, captured by a fluorescence microscope, of a plurality of individual separated compartments detected by the CRISPR/Cas system using a well plate with a nucleic acid as the target substance. A particle was used as the target capturing substance. The left is a bright field image, and the right is a fluorescence image. The particle can be seen in the bright field image in FIG. 3. In the fluorescence image, the fluorescence derived from the detection reagent can be seen. Further, in the well shining at the center of a left edge of the fluorescence image in FIG. 3, no particles can be confirmed in the bright field image. In other words, the target substance dissociated from the particle and was accidentally trapped in the individual separated compartment, where it reacted and emitted fluorescence.

### <Detection Process>

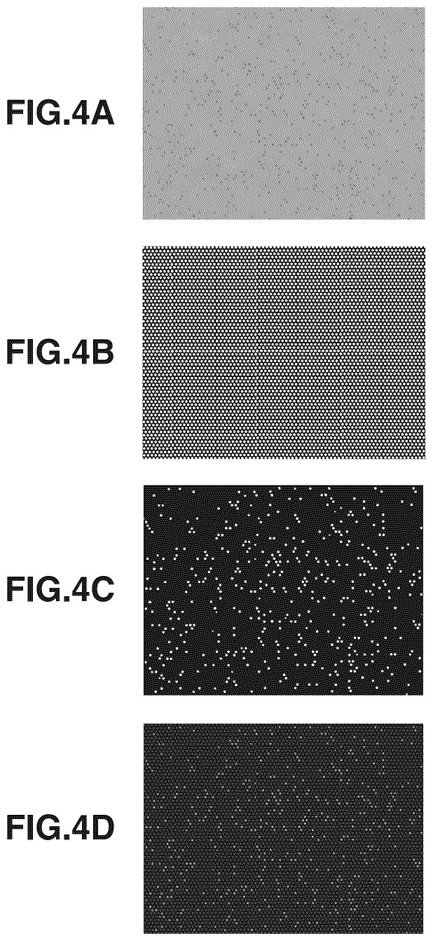

The information processing method according to the present exemplary embodiment includes the detection process for detecting a position of the individual separated compartment and a position of the target capturing substance. In the detection process, a position image of the individual separated compartment and a position image of the target capturing substance are generated. Either a bright field image as illustrated in FIG. 4A or an image from which signal information indicating the presence of the target substance and the individual separated compartment is acquired is used. Accordingly, the position image of the individual separated compartment in which a position of each individual separated compartment is detected as illustrated in FIG. 4B and the position image of the individual separated compartment in which the target capturing substance is detected that detects a position of the individual separated compartment containing the target capturing substance as illustrated in FIG. 4D can be generated. Here, the position image of the individual separated compartment in which the target capturing substance is detected is, for example, an image of an extracted well containing a particle.

A following method may be considered for a method for generating a position image of the individual separated compartment according to the present exemplary embodiment.

For example, in a case where the aggregate is a well plate, wells, which are the individual separated compartments, are regularly arranged, so that a method for generating position images of the individual separated compartments in advance and adjusting a translation direction and an angle using template matching or the like may be used. It is also possible to use a method for using edge detection of a Sobel filter or the like to emphasize an outer periphery of the individual separated compartment and detecting a circle by performing Hough transformation or the like.

A mask image extracting the target capturing substance is generated as illustrated in FIG. 4C and is overlapped on the position image of the individual separated compartment, so that the position image of the individual separated compartment in which the target capturing substance is detected can be acquired as illustrated in FIG. 4D.

### <Calculation Process>

In the calculation process according to the present exemplary embodiment, a luminance value of each individual separated compartment is calculated while distinguishing between the individual separated compartment in which the target capturing substance is detected and the individual separated compartment in which the target capturing substance is not detected.

According to the present exemplary embodiment, in the calculation process, calculation can be performed based on the position image of the individual separated compartment acquired in the detection process and the position image of the individual separated compartment in which the target capturing substance is detected. The luminance value refers to luminance in the bright field image and intensity of the signal (for example, fluorescence intensity) indicating the presence of the target substance in the image from which the signal information indicating the presence of the target substance can be acquired. The luminance value is a statistical value of pixel values within the individual separated compartment and may be an average, median, minimum, or maximum value.

The calculation process according to the present exemplary embodiment may include a process for determining whether the compartment is labelled as positive or negative based on a relationship between a predetermined threshold value and the luminance value of the individual separated compartment in which the target capturing substance is detected and a relationship between a predetermined threshold value and the luminance value of the individual separated compartment in which the target capturing substance is not detected. Specifically, whether a negative individual separated compartment or a positive individual separated compartment is determined using the respective predetermined threshold values with respect to the calculated luminance value (group) of the individual separated compartment in which the target capturing substance is detected and the calculated luminance value (group) of the individual separated compartment in which the target capturing substance is not detected. The determination is sometimes referred to as the determination between negative and positive. The positive individual separated compartment is an individual separated compartment that indicates that it contains the target substance, and the negative individual separated compartment is an individual separated compartment that indicates that it does not contain the target substance. In other words, the individual separated compartments are classified into following four types:
the positive individual separated compartment in which the target capturing substance is detected;
the negative individual separated compartment in which the target capturing substance is detected;
the positive individual separated compartment in which the target capturing substance is not detected; and
the negative individual separated compartment in which the target capturing substance is not detected.

Whether it is the positive individual separated compartment or the negative individual separated compartment can be determined, for example, by setting a threshold value for information of signal intensity (signal intensity and luminance value) in the image from which the signal information indicating the presence of the target substance can be acquired. For example, the threshold value may be automatically determined from the luminance value of each individual separated compartment using Otsu's method or the like, or the threshold value may be determined from variation in the signal intensity based on the aggregate that does not contain the target substance. The threshold value may be determined based on the signal intensity of the individual separated compartment that does not contain the target capturing substance.

It is conceivable that a reason why the luminance value of the individual separated compartment is high even though it does not contain the target capturing substance is that the target substance is released from the target capturing substance at a certain rate in the liquid and is accidentally isolated in the individual separated compartment that does not contain the target capturing substance. A release model in a case of an antigen-antibody reaction can be expressed as a following Expression (1). Here, Ab is an antibody, Ag is an antigen, AbAg indicates a state in which the antibody and the antigen bind to each other.

$$A b + A g ↔ AbAg \qquad ( 1 )$$

It can be seen from the above-described Expression (1) that the antibody and the antigen bind and dissociate at a certain rate. The rate of binding and dissociating is determined by a following Equation (2).

$$Kd = [ A b ] [ Ag ] / [ AbAg ] \qquad ( 2 )$$

Here, [Ab] is a concentration of the antibody, [Ag] is a concentration of the antigen, [AbAg] is a concentration of the bound antibody and antigen, and Kd is a disassociation constant. The disassociation constant is different for each antibody and, according to Equation (2), the bound rate depends on the concentrations of the antibody and the antigen.

The dissociated target substance may enter the individual separated compartment that contains the target capturing substance or the individual separated compartment that does not contain the target capturing substance. According to a conventional method, only the individual separated compartment in which the target capturing substance is present is a measurement target. However, according to the present exemplary embodiment, the individual separated compartment that does not contain the target capturing substance is also a measurement target to calculate the concentration of the target substance.

In order to calculate a measurement value, it is necessary to separate the individual separated compartment in which the target capturing substance is detected and the individual separated compartment in which the target capturing substance is not detected. First, in a case of the individual separated compartment in which the target capturing substance is detected, an average positive rate (APR), which indicates a rate of positive compartments among measurement target compartments, is calculated using Equation (3).

$$APRb = BP / ( BP + B N ) \qquad ( 3 )$$

Here, APRb is an APR of the individual separated compartment in which the target capturing substance is detected, BP is the number of positive individual separated compartments in which the target capturing substance is detected, and BN is the number of negative individual separated compartments in which the target capturing substance is detected.

Similarly, in a case of the individual separated compartment in which the target capturing substance is not detected, an APR is calculated using Equation (4).

$$APRw = WP / ( WP + WN ) \qquad ( 4 )$$

Here, APRw is an APR of the individual separated compartment that does not contain the target capturing substance, WP is the number of positive individual separated compartments in which the target capturing substance is not detected, and WN is the number of negative individual separated compartments in which the target capturing substance is not detected.

Next, an APR(P(k)) is calculated from each of the APRs of the individual separated compartments in which the target capturing substance is detected and the individual separated compartments in which the target capturing substance is not detected. In a case where the sample contains a large number of target substances, one individual separated compartment may contain two or more target substances. Thus, the number of target substances may not match the number of individual separated compartments that generate signals. For an above-described reason, it is desirable to calculate the concentration of the target substance by a calculation considering a Poisson distribution. In the Poisson distribution, if an average number of target substances per individual separated compartment is λ, a rate P(k) of wells that generate signals can be expressed using a following Equation (5).

$$P ( k \vert λ ) = ( \frac{λ k}{k !} ) {e}^{- λ} ( k = 0 , 1 , 2 , … ) \qquad ( 5 )$$

The rate P(k) can be calculated from the number of individual separated compartments that generate signals, and λ that is the number of target substances can be calculated.

A concentration C of the target substance in the liquid can be calculated using Equation (6), where λb is the average number of target substances in the individual separated compartment in which the target capturing substance is detected, and λw is the average number of target substances in the individual separated compartment in which the target capturing substance is not detected.

$$C = λ b \times bead + λ w \times \{ ( L a - Lb ) / Lw \} \qquad ( 6 )$$

Here, bead is a total number of target capturing substances contained in the liquid, La is a volume of the liquid, Lw is a individual separated compartment volume, Lb is a total volume of the individual separated compartments in which the target capturing substance is detected.

The target substance dissociated from the target capturing substance can be a detection target by using the information processing method according to the present exemplary embodiment, so that measurement sensitivity and measurement accuracy can be improved. Accordingly, measurement stability can be improved in measurement in a case where the target substance is at a low concentration, and a lower detection limit can be expanded.

In addition, the concentrations of the dissociated target substance and the bound target substance are known, if the disassociation constant of the antibody is known, it is possible to confirm whether the assay is going well using Equation (2).

Further, the present disclosure can provide a program for executing the information processing method according to the above-described present exemplary embodiment.

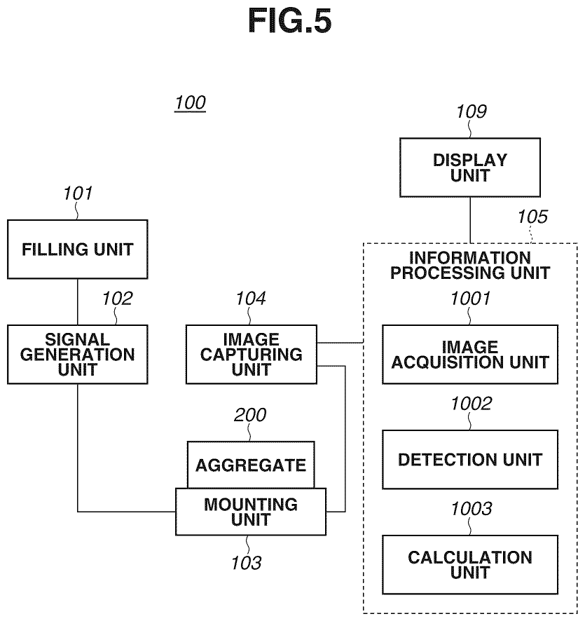

FIG. 5 illustrates an information processing system and a calculation system according to the present exemplary embodiment.

A calculation apparatus 100 according to the present exemplary embodiment includes a mounting unit 103, an image capturing unit 104, and an information processing unit 105. The information processing unit 105 includes an image acquisition unit 1001, a detection unit 1002, and a calculation unit 1003.

The mounting unit 103 mounts the aggregate 200 thereon. The mounting unit 103 may include a fixing unit suitable to mount the aggregate 200 thereon. The image capturing unit 104 captures an image including a plurality of individual separated compartments included in the aggregate as an object. The image capturing unit 104 is connected to the information processing unit 105.

The calculation system according to the present exemplary embodiment can further include a filling unit 101, a signal generation unit 102, and a display unit 109. The filling unit 101 can include a robotic arm, a pipette, a device for deaerating, and a pumping device for filling the liquid containing the target substance into the individual separated compartment. The signal generation unit 102 includes an incubator, a stirrer, and a permeation device so that the individual separated compartment filled with the liquid containing the target substance generates a signal. The display unit 109 includes a monitor of a personal computer. In the calculation system according to the present exemplary embodiment, each unit may be included in a single device or separate devices. For example, the information processing unit 105 may be provided on a cloud, and information may be exchanged by a communication unit.

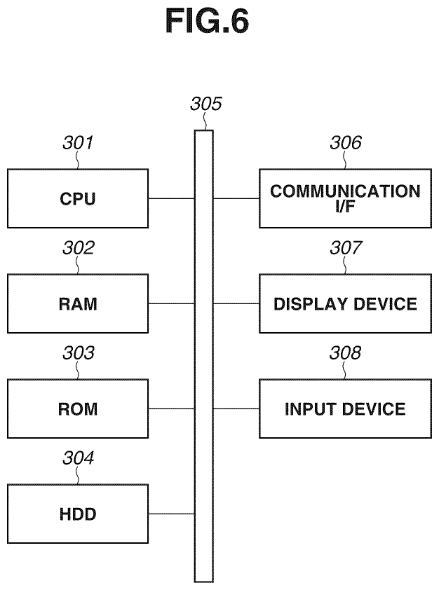

A hardware configuration of the information processing unit 105 according to the present exemplary embodiment is described with reference to FIG. 6.

The information processing unit 105 according to the present exemplary embodiment has a function of a computer. For example, the information processing system may be integrally configured with a desktop personal computer (PC), a laptop PC, a tablet PC, a smartphone, or the like. The information processing unit 105 may have a function of controlling operations of the filling unit 101, the signal generation unit 102, and the image capturing unit 104 according to a predetermined program.

The information processing unit 105 according to the present exemplary embodiment includes a central processing unit (CPU) 301, a random access memory (RAM) 302, a read only memory (ROM) 303, and a hard disk drive (HDD) 304 in order to realize a function as a computer that performs calculation and storage. The information processing unit 105 further includes a communication interface (I/F) 306, a display device 307, and an input device 308. The CPU 301, the RAM 302, the ROM 303, the HDD 304, the communication I/F 306, the display device 307, and the input device 308 are connected to each other via a bus 305. The display device 307 and the input device 308 may be connected to the bus 305 via a drive device (not illustrated) for driving these devices.

FIG. 6 illustrates each unit configuring the information processing unit 105 as an integrated device, but a part of the functions may be configured with an external device. For example, the display device 307 and the input device 308 may be external devices separate from a part that configures the function of the computer including the CPU 301 and others.

According to the present exemplary embodiment, the CPU 301 performs a predetermined operation according to a program stored in the RAM 302, the HDD 304, or the like and also has a function of controlling each unit in the information processing unit 105. The RAM 302 is configured with a volatile storage medium and provides a temporary memory area necessary for the operation of the CPU 301. The ROM 303 is configured with a non-volatile storage medium and stores necessary information such as a program used in the operation of the information processing unit 105. The HDD 304 is a storage device that is configured with a non-volatile storage medium and stores information regarding the number and positions of individual separated compartments, fluorescence intensity, and the like.

According to the present exemplary embodiment, the communication I/F 306 is a communication interface based on standards such as Wi-Fi® and 4G and is a module for communicating with other devices. The display device 307 is a liquid crystal display, an organic light emitting diode (OLED) display, or the like and is used to display a moving image, a still image, a text, and the like. The input device 308 includes a button, a touch panel, a keyboard, and a pointing device and is used by a user to operate the information processing unit 105. The display device 307 and the input device 308 may be integrally formed as a touch panel.

The hardware configuration illustrated in FIG. 6 is an example, and a device other than the above-described ones may be added, or a part of the devices may not be provided. Further, a part of the devices may be replaced with another device having a similar function. Furthermore, a part of the functions may be provided by another device via a network, and the functions included in the present exemplary embodiment may be realized by distributing them among a plurality of devices. For example, the HDD 304 may be replaced with a solid state drive (SSD) using a semiconductor element such as a flash memory or may be replaced with a cloud storage.

According to the present exemplary embodiment, the CPU 301 loads a program stored in the ROM 303 or the like to the RAM 302 and executes it, thereby realizing the functions of the image acquisition unit 1001, the detection unit 1002, and the calculation unit 1003. Further, the CPU 301 controls the display device 307 to realize the function of the display unit 109. Furthermore, the CPU 301 controls the HDD 304 to realize a function of a storage unit.

The information processing system according to the present exemplary embodiment includes the above-described information processing unit 105.

The present disclosure can also be realized by executing the following processing. More specifically, a program for realizing one or more functions of the above-described exemplary embodiment is supplied to a system or an apparatus via a network or a storage medium and one or more processors of a computer of the system or the apparatus read and execute the program. Further, the present disclosure can also be realized by a circuit (for example, an application specific integrated circuit (ASIC)) that realizes one or more functions.

The above-described present disclosure includes the following methods, program, and configurations.

### (Method 1)

A method for processing information for detecting a target substance includes acquiring an image including, as an object, a plurality of individual separated compartments that contains: 1) at least one of the target substance and a target capturing substance that captures the target substance, or 2) neither the target substance nor the target capturing substance, detecting presence or absence of the target capturing substance in each of the individual separated compartments in the image, and calculating an amount or a concentration of the target substance based on information regarding 1) the number of the individual separated compartments in which the target capturing substance is detected and 2) the number of compartments in which the target substance is detected among the individual separated compartments in which the target capturing substance is detected and information regarding 3) the number of the individual separated compartments in which the target capturing substance is not detected and 4) the number of compartments in which the target substance is detected among the individual separated compartments in which the target capturing substance is not detected.

### (Method 2)

There is provided the method according to method 1, wherein the individual separated compartment is a well or a droplet.

### (Method 3)

There is provided the method according to method 1 or 2, wherein the target capturing substance contains a particle.

### (Method 4)

There is provided the method according to method 3, wherein the particle contains a magnetic particle.

### (Method 5)

There is provided the method according to method 3 or 4, wherein a particle diameter of the particle is 1 μm or more and 10 μm or less.

### (Method 6)

There is provided the method according to any one of methods 1 to 5, wherein the target capturing substance includes at least one of a nucleic acid, an antibody, an antigen, an enzyme, and an enzyme substrate.

### (Method 7)

There is provided the method according to any one of methods 1 to 6, wherein the target substance includes at least one of a nucleic acid, an antibody, an antigen, an enzyme, and an enzyme substrate.

### (Method 8)

There is provided the method according to any one of methods 1 to 7, wherein the image includes at least one of a bright field image and a fluorescence image.

### (Method 9)

There is provided the method according to any one of methods 1 to 8, wherein a volume of the individual separated compartment is 0.1 fL or more and 1000 fL or less.

### (Method 10)

There is provided the method according to any one of methods 1 to 9, wherein the calculating includes determining a negative or positive label based on a relationship between a predetermined threshold value and a luminance value of the individual separated compartments in which the target capturing substance is detected and a relationship between a predetermined threshold value and a luminance value of the individual separated compartment in which the target capturing substance is not detected.

### (Method 11)

There is provided the method according to any one of methods 1 to 10, wherein the calculating includes calculating an amount or a concentration of the target substance using an average number of target substances in the individual separated compartment in which the target capturing substance is detected and an average number of target substances in the individual separated compartment in which the target capturing substance is not detected.

### (Storage Medium)

There is provided a storage medium storing a program for executing the method according to any one of methods 1 to 11.

### (Configuration 1)

A calculation system includes a mounting unit configured to mount an aggregate that includes a plurality of individual separated compartments thereon, an image capturing unit configured to capture an image that includes the plurality of individual separated compartments as an object, and an information processing unit, wherein the information processing unit includes an image acquisition unit configured to acquire an image that includes, as an object, a plurality of individual separated compartments that contains: 1) at least one of a target substance and a target capturing substance that captures the target substance, or 2) neither the target substance nor the target capturing substance, a detection unit configured to detect presence or absence of the target capturing substance in each of the individual separated compartments in the image, and a calculation unit configured to calculate an amount or a concentration of the target substance based on information regarding 1) the number of the individual separated compartments in which the target capturing substance is detected and 2) the number of compartments in which the target substance is detected among the individual separated compartments in which the target capturing substance is detected and information regarding 3) the number of the individual separated compartments in which the target capturing substance is not detected and 4) the number of compartments in which the target substance is detected among the individual separated compartments in which the target capturing substance is not detected.

### (Configuration 2)

An information processing system including an information processing unit configured to process an image that includes a plurality of individual separated compartments as an object, wherein the information processing unit includes an image acquisition unit configured to acquire an image that includes, as an object, a plurality of individual separated compartments that contains: 1) at least one of a target substance and a target capturing substance that captures the target substance, or 2) neither the target substance nor the target capturing substance, a detection unit configured to detect presence or absence of the target capturing substance in each of the individual separated compartments in the image, and a calculation unit configured to calculate an amount or a concentration of the target substance based on information regarding 1) the number of the individual separated compartments in which the target capturing substance is detected and 2) the number of compartments in which the target substance is detected among the individual separated compartments in which the target capturing substance is detected and information regarding 3) the number of the individual separated compartments in which the target capturing substance is not detected and 4) the number of compartments in which the target substance is detected among the individual separated compartments in which the target capturing substance is not detected.

According to the present disclosure, accuracy of calculating a concentration of a target substance can be improved.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2023-201983, filed Nov. 29, 2023, which is hereby incorporated by reference herein in its entirety.

What is claimed is:

1. A method for processing information for detecting a target substance, the method comprising:
acquiring an image including, as an object, a plurality of individual separated compartments that contain: 1) at least one of the target substance and a target capturing substance that captures the target substance, or 2) neither the target substance nor the target capturing substance;
detecting the presence or absence of the target capturing substance in each of the individual separated compartments in the image; and
calculating an amount or a concentration of the target substance based on information regarding 1) the number of the individual separated compartments in which the target capturing substance is detected and 2) the number of compartments in which the target substance is detected among the individual separated compartments in which the target capturing substance is detected and information regarding 3) the number of the individual separated compartments in which the target capturing substance is not detected and 4) the number of compartments in which the target substance is detected among the individual separated compartments in which the target capturing substance is not detected.

2. The method according to claim 1, wherein the individual separated compartment is a well or a droplet.

3. The method according to claim 1, wherein the target capturing substance contains a particle.

4. The method according to claim 3, wherein the particle contains a magnetic particle.

5. The method according to claim 3, wherein a particle diameter of the particle is 1 μm or more and 10 μm or less.

6. The method according to claim 1, wherein the target capturing substance includes at least one of a nucleic acid, an antibody, an antigen, an enzyme, and an enzyme substrate.

7. The method according to claim 1, wherein the target substance includes at least one of a nucleic acid, an antibody, an antigen, an enzyme, and an enzyme substrate.

8. The method according to claim 1, wherein the image includes at least one of a bright field image and a fluorescence image.

9. The method according to claim 1, wherein a volume of the individual separated compartment is 0.1 fL or more and 1000 fL or less.

10. The method according to claim 1, wherein the calculating includes determining a negative or positive label based on a relationship between a predetermined threshold value and a luminance value of the individual separated compartments in which the target capturing substance is detected and a relationship between a predetermined threshold value and a luminance value of the individual separated compartment in which the target capturing substance is not detected.

11. The method according to claim 1, wherein the calculating includes calculating an amount or a concentration of the target substance using an average number of target substances in the individual separated compartment in which the target capturing substance is detected and an average number of target substances in the individual separated compartment in which the target capturing substance is not detected.

12. A storage medium storing a program for executing the method according to claim 1.

13. A calculation system comprising:
a mounting unit configured to mount an aggregate that includes a plurality of individual separated compartments thereon;
an image capturing unit configured to capture an image that includes the plurality of individual separated compartments as an object; and
an information processing unit,
wherein the information processing unit includes:
an image acquisition unit configured to acquire an image that includes, as an object, a plurality of individual separated compartments that contains: 1) at least one of a target substance and a target capturing substance that captures the target substance, or 2) neither the target substance nor the target capturing substance;
a detection unit configured to detect presence or absence of the target capturing substance in each of the individual separated compartments in the image; and
a calculation unit configured to calculate an amount or a concentration of the target substance based on information regarding 1) the number of the individual separated compartments in which the target capturing substance is detected and 2) the number of compartments in which the target substance is detected among the individual separated compartments in which the target capturing substance is detected and information regarding 3) the number of the individual separated compartments in which the target capturing substance is not detected and 4) the number of compartments in which the target substance is detected among the individual separated compartments in which the target capturing substance is not detected.

14. An information processing system including an information processing unit configured to process an image that includes a plurality of individual separated compartments as an object,
wherein the information processing unit includes:
an image acquisition unit configured to acquire an image that includes, as an object, a plurality of individual separated compartments that contains: 1) at least one of a target substance and a target capturing substance that captures the target substance, or 2) neither the target substance nor the target capturing substance;
a detection unit configured to detect presence or absence of the target capturing substance in each of the individual separated compartments in the image; and
a calculation unit configured to calculate an amount or a concentration of the target substance based on information regarding 1) the number of the individual separated compartments in which the target capturing substance is detected and 2) the number of compartments in which the target substance is detected among the individual separated compartments in which the target capturing substance is detected and information regarding 3) the number of the individual separated compartments in which the target capturing substance is not detected and 4) the number of compartments in which the target substance is detected among the individual separated compartments in which the target capturing substance is not detected.
